(19) United States
(12) Patent Application Publication
(10) Pub. No.: US 2009/0075992 A1
(43) Pub. Date: Mar. 19, 2009

(54) Pteridine Derivatives as Nitric Oxide Synthase Activators

(75) Inventors: Roger Wadsworth, Glasgow (GB); Colin Suckling, Glasgow (GB); Colin Gibson, Glasgow (GB)

Correspondence Address: ALSTON & BIRD LLP, BANK OF AMERICA PLAZA, 101 SOUTH TRYON STREET, SUITE 4000, CHARLOTTE, NC, 28280-4000, US

(73) Assignee: University of Strathclyde

(21) Appl. No.: 12/207,631

(22) Filed: Sep. 10, 2008

Related U.S. Application Data
(63) Continuation-in-part of application No. 11/322,914, filed on Dec. 30, 2005.
(60) Provisional application No. 60/642,013, filed on Jan. 7, 2005.

(51) Int. Cl. A61K 31/4985; A61K 31/5383; A61P 3/00; A61P 7/00

(52) U.S. Cl. 514/230.5; 514/249; 514/250

(57) ABSTRACT

The present invention relates to the use of pteridine derivatives as nitric oxide synthase activators. In particular, the derivatives find use in the treatment of diseases associated with endothelial dysfunction such as cardiovascular diseases.

### CROSS-REFERENCE TO RELATED APPLICATIONS

This continuation-in-part application claims the benefit of U.S. application Ser. No. 11/322,914, filed Dec. 30, 2005 which claims the benefit of U.S. Provisional Application No. 60/642,013, filed Jan. 7, 2005, which is incorporated by reference herein in its entirety.

### FIELD OF THE INVENTION

The present invention relates to the use of pteridine derivatives as nitric oxide synthase activators. In particular, the derivatives find use in the treatment of diseases associated with endothelial dysfunction such as cardiovascular diseases.

### BACKGROUND OF THE INVENTION

Nitric oxide (NO) has a vital role in cardiovascular physiology. It is a major mediator that maintains normal blood pressure, distribution of blood flow, regulation of platelet aggregation and leukocyte adhesion, and remodelling of blood vessel structure.

NO is the chief signalling chemical produced by the vascular endothelium, and the major cardiovascular diseases are all associated with disturbed endothelial function. This dysfunction significantly contributes to the progression of disease.

Deficiency of NO has been found in atherosclerosis, diabetes and hyperlipidaemia. The lack of NO in the cardiovascular system explains many of the characteristic features of these conditions, such as leukocyte infiltration, vascular spasm, neointimal hypertrophy, and hypercoagulability. Diabetes is associated with markedly increased incidence of atherosclerosis and increased risk of myocardial ischaemia, stroke and peripheral vascular disease. Many patients with type 2 diabetes also have hyperlipidaemia, which is an additional risk factor for atherosclerosis. Thus, the inter-related conditions of diabetes, atherosclerosis and hyperlipidaemia are all associated with endothelial dysfunction and NO deficiency. Patients generally have a poor prognosis.

NO is formed by the enzyme NO synthase in the endothelial cells. In its normal state, each molecule of NO synthase contains one molecule of tetrahydrobiopterin as an essential co-factor. The importance of tetrahydrobiopterin is clearly demonstrated if purified NO synthase enzyme is stripped of its tetrahydrobiopterin co-factor. This destroys its enzymatic activity, although replacing the tetrahydrobiopterin can restore this (1).

Tetrahydrobiopterin has an essential role in the redox reactions required for formation of NO, and it may have additional functions in the enzyme, such as stabilising the association of enzyme monomers to form dimers that are necessary for activity. If NO synthase is present but dysfunctional, it can generate superoxide in place of NO, which is doubly harmful since superoxide not only consumes NO but also forms the cytotoxic product peroxynitrite.

Blood vessels removed from diabetic patients have impaired NO formation but elevated superoxide formation, and the administration of tetrahydrobiopterin to the artery rings restores endothelium function to normal (2). Sepiapterin (the precursor in tetrahydrobiopterin synthesis) also restores normal endothelium-dependent relaxation in coronary arterioles obtained from patients with atherosclerosis, while sepiapterin has no effect in arterioles from non-atherosclerotic patients (3).

The critical importance of tetrahydrobiopterin in disease is further revealed by animal studies. Two independent studies with animal models of diabetes demonstrated that diabetic animals had reduced levels of tetrahydrobiopterin; there was impaired NO formation and consequent loss of vascular responsiveness (4, 5). Moreover, restoration of normal tetrahydrobiopterin levels by overexpression of the gene for the synthesis of tetrahydrobiopterin restored normal NO formation and endothelial function in the diabetes model (6).

In an animal model of atherosclerosis induced by hyperlipidaemia, amounts of tetrahydrobiopterin in artery tissues were reduced to extremely low levels, but could be normalised by administration of sepiapterin, which is the precursor for tetrahydrobiopterin (7). In the

ApoE−/− knock-out mouse, which is another animal model of atherosclerosis, administration of tetrahydrobiopterin prevented the development of atherosclerotic lesions (8). Moreover, techniques that reduce availability of tetrahydrobiopterin in experimental models, such as treatment with an inhibitor of the enzyme GTP cyclohydrolase (the rate limiting enzyme in the synthesis of tetrahydrobiopterin), impaired flow-induced NO formation by arterioles similar to that seen in experimental diabetes, which was restored to normal by sepiapterin (9).

Recent reviews have documented the evidence for tetrahydrobiopterin deficiency in human patients with diabetes and atherosclerosis, and the potential that this has as a drug target (10-12). One current hypothesis is that oxidative stress attacks tetrahydrobiopterin, leading to loss of NO synthase activity and NO, exacerbated by the switch of NO synthase from NO formation to superoxide formation, leading to both the immediate and long-term deterioration of artery function that is characteristic of atherosclerosis (10-12).

Tetrahydrobiopterin itself is available for clinical use, although its poor bioavailability means that it has to be given by injection. However, there have already been several clinical studies involving administration of tetrahydrobiopterin to patients whose endothelium function is reduced as a result of cardiovascular disease. Intra-arterial infusion of tetrahydrobiopterin to diabetic patients restored endothelium-dependent vasodilatation to normal; however, intra-arterial infusion of tetrahydrobiopterin to the control group of subjects was without effect (13). Similar results were obtained when intra-arterial 5-methyltetrahydrofolate was substituted for tetrahydrobiopterin (14).

Hyperglycaemia, which occurs in diabetes, impairs endothelium-dependent relaxation in human subjects, and this is reversed by intra-arterial administration of tetrahydrobiopterin (15). Intra-arterial infusion of tetrahydrobiopterin restores endothelium-dependent vasodilatation in patients with hypercholesterolaemia (16). Acetylcholine, which is normally an endothelium-dependent vasodilator, is a vasoconstrictor in hypercholesterolaemic patients; however, intra-arterial infusion of tetrahydrobiopterin restores endothelium-dependent vasodilatation to normal (17).

In diabetes and atherosclerosis, damage to the arteries is localised and the major symptoms are often linked to one or a small number of atherosclerostic plaques.

The present invention seeks to obviate and/or mitigate one or more of the above-mentioned problems, in particular the disadvantages of tetrahydrobiopterin, and its natural precursors, sepiapterin and biopterin.

Accordingly, it is an object of the present invention to provide use of a pteridine derivative in a method to enhance nitric oxide formation by nitric oxide synthase.

It is a further object of the present invention to provide use of a pteridine derivative in a method to reverse a local deficiency of nitric oxide.

It is yet a further object of the present invention to provide use of a pteridine derivative in a method to locally reverse a nitric oxide deficiency in dysfunctional endothelium.

It is another object of the present invention to provide use of a pteridine derivative in a method to treat a disease associated with endothelial dysfunction.

It is yet another object of the present invention to provide use of a pteridine derivative in a method to selectively reverse a local deficiency of nitric oxide.

### SUMMARY OF INVENTION

According to a first aspect of the present invention, there is provided a method of enhancing nitric oxide formation by nitric oxide synthase, comprising administering to a subject in need thereof a compound of formula (I):

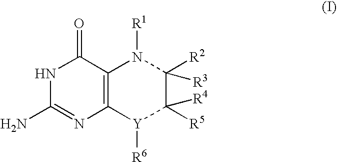

or pharmaceutically acceptable derivatives and/or salts thereof, wherein,

Y is an oxygen or a nitrogen atom;

R² and R⁵ are independently hydrogen, unsubstituted or substituted alkyl, unsubstituted or substituted alkenyl, unsubstituted or substituted aryl, hydroxyl, amino, halo, alkanoyl, alkyl carboxy, sulfonyl and hydroxyimino;

R³ and R⁴ are independently hydrogen, unsubstituted or substituted alkyl, unsubstituted or substituted alkenyl, unsubstituted or substituted aryl, hydroxyl, amino, halo, alkanoyl, alkyl carboxy, sulfonyl and hydroxyimino,

or R³ and R⁴ taken together with the ring carbons to which they are bonded, form an unsubstituted or substituted carbocyclic ring;

the dashed lines are independently a carbon-carbon single bond, wherein R¹ and R⁶ are hydrogen, or the dashed lines are independently a carbon-carbon double bond and the groups R¹ and R², and/or R⁵ and R⁶ associated with the carbon-carbon double bond are absent, with the proviso that when Y is an oxygen atom, R⁶ is absent and the dashed line attached to Y is a single bond.

According to a second aspect of the present invention, there is provided a method for the treatment of a disease associated with a deficiency of nitric oxide, comprising administering to a subject in need thereof, a compound according to formula (I).

According to a third aspect of the present invention, there is provided a method for the treatment of a disease associated with endothelial dysfunction comprising administering to a subject in need thereof, a compound according to formula (I).

According to a fourth aspect of the present invention, there is provided a method for selectively increasing a local amount of nitric oxide, comprising administering to a subject in need thereof a compound according to formula (I).

For example a subject in need thereof may be one in which there is a local deficiency of nitric oxide. Such local deficiency of nitric oxide may therefore be rectified by causing a release of or causing an increase of the amount of nitric oxide at a selected location.

The release or increase may be caused in one or more tissue types or at particular regions, e.g. at one or more veins or arteries. The release or increase may be caused at a selected location of a vein or artery e.g. at site of damage or injury of a vein or artery.

Preferred compounds of formula (I) are those in which R², R³, R⁴ and R⁵ are independently unsubstituted or substituted alkyl, alkenyl, alkanoyl or carboxy.

Typical alkyl and alkenyl groups are those having from 1 to 24 carbon atoms.

Preferred alkyl groups include lower alkyl, i.e. C₁-C₁₀, preferably C₁-C₆, e.g. C₁-C₄ alkyl.

Preferred alkenyl groups include lower alkenyl, i.e. C₁-C₁₀, preferably C₁-C₆, e.g. C₁-C₄ alkyl.

Substituents on the alkyl and alkenyl groups may be chosen from hydroxy, amino, carboxy, halo, sulfonyl and unsubstituted or substituted aryl.

Typical aryl groups are those having 6, 8, 10 or 14 carbon atoms. For example the aryl group may be a substituted or unsubstituted phenyl or napthyl group.

A preferred aryl group is substituted or unsubstituted phenyl.

Substituents on the aryl group may be chosen from alkyl, hydroxy, amino, carboxy and halo.

Herein, halo refers to fluoro, chloro, bromo or iodo.

Particular examples of substituted alkyl groups encompassed in the present invention include hydroxy methyl, benzyl and phenethyl.

A particular preferred substituted alkenyl group is 2-(halophenyl)-vinyl e.g., 2-(4-chlorophenyl)-vinyl.

Preferred alkanoyl groups include ketones and aldehydes.

Alternatively, R³ and R⁴ form a carbocyclic ring with the carbons to which they are attached, which may be a 5 to 7 saturated or unsaturated ring e.g. a cyclopentane or cyclohexane ring may be formed.

Preferably, R⁴ and R⁵ are independently either hydrogen or lower alkyl.

A preferred compound is one in which both of the dashed lines are double bonds and R¹, R², R⁵, and R⁶ are absent, R² is hydroxymethyl i.e. —CH₂OH, and R⁴ is hydrogen.

Another preferred compound is one in which one of the dashed lines is a double bond and

R¹ and R² (associated with that double bond) are absent, R³ is acetyl i.e. —C(O)CH₃, R⁴ and R⁵ are methyl, and R⁶ is hydrogen.

Yet another preferred compound is one in which one of the dashed lines is a double bond and R¹ and R² (associated with that double bond) are absent, R³ is 2-(4-chlorophenyl)-vinyl i.e. —CH═CH-4-Chlorophenyl, R⁴ and R⁵ are methyl, and R⁶ is hydrogen.

The present invention is based on the observation that diabetes, atherosclerosis and hyperlipidaemia are associated with endothelial dysfunction and deficiency of nitric oxide, which is formed by the enzyme nitric oxide synthase which contains tetrahydrobiopterin as a co-factor.

Tetrahydrobiopterin has poor bioavailability and must be administered by injection and the present invention seeks to overcome this by providing methods which make use of compounds intended to have improved bioavailability and which do not necessarily need to be administered by injection. For example, compared to tetrahydrobiopterin, the compounds may have improved oral bioavailability, improved cell penetration, longer duration of action, greater potency and/or greater specificity for particular isoforms of nitric oxide synthase.

The methods of the present invention may be used to treat diabetes, atherosclerosis, hyperlipidaemia and other disease states which are directly or indirectly associated with a lack of nitric oxide, e.g. arterial high blood pressure, haemostasis disorders, coronary heart disease and erectile dysfunction.

Without wishing to be bound by theory, it is believed that the compounds used in the methods of the present invention activate nitric oxide synthase so that nitric oxide is produced.

Accordingly, according to a fifth aspect of the present invention there is provided a method of activating nitric oxide synthase, comprising administering to a subject in need thereof a compound of formula (I).

For the avoidance of doubt, the methods described herein extend to the uses of the compounds of formula (I) in such methods, for example for the preparation of medicaments for use in the methods.

The subject to be treated may be a human or non-human animal.

The term nitric oxide synthase referred to herein includes any of the types of nitric oxide synthase (NOS) found in living systems. For example several isoforms exist such as endothelial nitric oxide synthase (eNOS), neuronal nitric oxide synthase (nNOS) and inducible nitric oxide synthase (iNOS).

According to a sixth aspect of the present invention there is provided a pharmaceutical formulation comprising a compound of formula (I) in combination with a pharmaceutically acceptable carrier therefor.

Thus, for use according to the present invention a compound of formula (I) is preferably presented as a pharmaceutical formulation, comprising a compound of formula (I) or a physiologically acceptable salt, ester or other physiologically functional derivative thereof (hereinafter referred to as “active compound”) together with one or more pharmaceutically acceptable carriers therefor and optionally other therapeutic and/or prophylactic ingredients. The carrier(s) must be acceptable in the sense of being compatible with the other ingredients of the formulation and not deleterious to the recipient thereof.

An active compound may conveniently be presented as a pharmaceutical formulation in unit dosage form. A convenient unit dose formulation contains an active compound in an amount of from 0.1 mg to 100 mg.

Pharmaceutical formulations include those suitable for oral, topical (including dermal, buccal and sublingual), rectal or parenteral (including subcutaneous, intradermal, intramuscular and intravenous), nasal and pulmonary administration e.g. by inhalation. The formulation may, where appropriate, be conveniently presented in discrete dosage units and may be prepared by any of the methods well known in the art of pharmacy. All methods include the step of bringing into association an active compound with liquid carriers or finely divided solid carriers or both and then, if necessary, shaping the product into the desired formulation.

Pharmaceutical formulations suitable for oral administration wherein the carrier is a solid are most preferably presented as unit dose formulations such as boluses, capsules or tablets each containing a predetermined amount of an active compound. A tablet may be made by compression or moulding, optionally with one or more accessory ingredients. Compressed tablets may be prepared by compressing in a suitable machine an active compound in a free-flowing form such as a powder or granules optionally mixed with a binder, lubricant, inert diluent, lubricating agent, surface-active agent or dispersing agent. Moulded tablets may be made by moulding an active compound with an inert liquid diluent. Tablets may be optionally coated and, if uncoated, may optionally be scored. Capsules may be prepared by filling an active compound, either alone or in admixture with one or more accessory ingredients, into the capsule shells and then sealing them in the usual manner. Cachets are analogous to capsules wherein an active compound together with any accessory ingredient(s) is sealed in a rice paper envelope. An active compound may also be formulated as dispersible granules, which may for example be suspended in water before administration, or sprinkled on food. The granules may be packaged e.g. in a sachet. Formulations suitable for oral administration wherein the carrier is a liquid may be presented as a solution or a suspension in an aqueous or non-aqueous liquid, or as an oil-in-water liquid emulsion.

Formulations for oral administration include controlled release dosage forms e.g. tablets wherein an active compound is formulated in an appropriate release—controlling matrix, or is coated with a suitable release—controlling film. Such formulation may be particularly convenient for prophylactic use.

Pharmaceutical formulations suitable for rectal administration wherein the carrier is a solid are most preferably presented as unit dose suppositories. Suitable carriers include cocoa butter and other materials commonly used in the art. The suppositories may be conveniently formed by admixture of an active compound with the softened or melted carrier(s) followed by chilling and shaping in moulds.

Pharmaceutical formulations suitable for parenteral administration include sterile solutions or suspensions of an active compound in aqueous or oleaginous vehicles. Injectible preparations may be adapted for bolus injection or continuous infusion. Such preparations are conveniently presented in unit dose or multi-dose containers which are sealed after introduction of the formulation until required for use. Alternatively, an active compound may be in powder form which is constituted with a suitable vehicle, such as sterile, pyrogen-free water, before use.

An active compound may also be formulated as long-acting depot preparations, which may be administered by intramuscular injection or by implantation e.g. subcutaneously or intramuscularly. Depot preparations may include, for example, suitable polymeric or hydrophobic materials, or ion-exchange resins. Such long-acting formulations are particularly convenient for prophylactic use.

Formulations suitable for pulmonary administration via the buccal cavity are presented such that particles containing an active compound and desirably having a diameter in the range 0.5 to 7 microns are delivered into the bronchial tree of the recipient.

As one possibility such formulations are in the form of finely comminuted powders which may conveniently be presented either in a pierceable capsule, suitably of, for example, gelatin, for use in an inhalation device, or alternatively as a self-propelling formulation comprising an active compound, a suitable liquid or gaseous propellant and optionally other ingredients such as a surfactant and/or a solid diluent. Suitable liquid propellants include propane and the chlorofluorocarbons, and suitable gaseous propellants include carbon dioxide. Self-propelling formulations may also be employed wherein an active compound is dispensed in the form of droplets of solution or suspension.

Such self-propelling formulations are analogous to those known in the art and may be prepared by established procedures. Suitably they are presented in a container provided with either a manually-operable or automatically functioning valve having the desired spray characteristics; advantageously the valve is of a metered type delivering a fixed volume, for example, 25 to 200 microlitres, upon each operation thereof.

As a further possibility an active compound may be in the form of a solution or suspension for use in an atomiser or nebuliser whereby an accelerated airstream or ultrasonic agitation is employed to produce a fine droplet mist for inhalation.

Formulations suitable for nasal administration include presentations generally similar to those described above for pulmonary administration. When dispensed such formulations should desirably have a particle diameter in the range 10 to 200 microns to enable retention in the nasal cavity; this may be achieved by, as appropriate, use of a powder of a suitable particle size or choice of an appropriate valve. Other suitable formulations include coarse powders having a particular diameter in the range 20 to 500 microns, for administration by rapid inhalation through the nasal passage from a container held close up to the nose, and nasal drops comprising 0.2 to 5% w/v of an active compound in aqueous or oily solution or suspension.

It should be understood that in addition to the aforementioned carrier ingredients the pharmaceutical formulations described above may include, as appropriate one or more additional carrier ingredients such as diluents, buffers, flavouring agents, binders, surface active agents, thickeners, lubricants, preservatives (including anti-oxidants) and the like, and substances included for the purpose of rendering the formulation isotonic with the blood of the intended recipient.

Therapeutic formulations for veterinary use may conveniently be in either powder or liquid concentrate form. In accordance with standard veterinary formulation practice, conventional water soluble excipients, such as lactose or sucrose, may be incorporated in the powders to improve their physical properties. Thus particularly suitable powders of this invention comprise 50 to 100% w/w, and preferably 60 to 80% w/w of the active ingredient(s), and 0 to 50% w/w and preferably 20 to 40% w/w of conventional veterinary excipients. These powders may either be added to animal feedstuffs, for example by way of an intermediate premix, or diluted in animal drinking water.

Liquid concentrates of this invention suitably contain a water-soluble compound of formula (I) or a salt-thereof and may optionally include a veterinarily acceptable water-miscible solvent, for example polyethylene glycol, propylene glycol, glycerol formal or such a solvent mixed with up to 30% v/v of ethanol. The liquid concentrates may be administered to the drinking water of animals.

### BRIEF DESCRIPTION OF DRAWINGS

FIGS. 1 and 2 respectively show the effects of the compound WSG1002 on rat aorta and rat pulmonary artery. WSG1002 had no effect on either rat aorta or rat pulmonary artery (▪) compared to control rings that received vehicle only (♦);

FIG. 3 shows relaxation by the compound WSG1002 of rat pulmonary arteries in which tetrahydrobiopterin biosynthesis was blocked by the GTP cyclohydrolase I inhibitor, DAHP. The upper trace shows the effect of WSG1002 in vehicle (MEM). The lower trace shows the substantial relaxation caused by WSG1002 in tetrahydrobiopterin depleted cells;

FIG. 4 shows relaxation by WSG1002 of rat aorta showing the significant relaxation produced at submicromolar concentrations and the lack of a significant effect of ammonium hydroxide in which WSG1002 was dissolved before addition to the test solution;

FIG. 5 shows the relaxant action of WSG1002 was prevented by incubation of the pulmonary artery ring with L-NAME (L-nitro-arginine methyl ester, 100 μM) which prevents NO formation from NO synthase. Note that WSG1002 produced no relaxation in contrast to FIG. 3. The same result was found in normal artery rings (endogenous tetrahydrobiopterin present) and after treatment with DAHP to deplete endogenous tetrahydrobiopterin;

FIG. 6 shows that tetrahydrobiopterin (BH4) had no relaxant effect on rat pulmonary artery either using normal artery rings (endogenous tetrahydrobiopterin present) or after treatment with DAHP to deplete endogenous tetrahydrobiopterin. Indeed, high concentrations of tetrahydrobiopterin (above 3 μM) caused contraction;

FIG. 7 shows low sensitivity macrophage assay (iNOS) in cells retaining the ability to biosynthesise tetrahydrobiopterin. No significant difference between controls and treated cells was evident;

FIG. 8 shows stimulation of nitric oxide synthesis in macrophages depleted of tetrahydrobiopterin by treatment with DAHP. The results between controls and treated cells are significantly different;

FIG. 9 shows nitric oxide production by tetrahydrobiopterin-depleted (DAHP) endothelial cells stimulated by solvent (DMSO, 0.5%) and WSG1002 at 30 μM added in DMSO solution;

FIG. 10 is a graph showing the effects of calcium ionophore-induced relaxation within normoxic rats. Normoxic SC refers to the animals treated with WSG1002. Values were expressed as means±SEM. Unpaired student's t-tests were used to make statistical comparisons of IC₅₀ values of the relaxation curves (*P≦0.05);

FIG. 11 is a graph showing the effects of calcium ionophore-induced relaxation within hypoxic rats. Hypoxic SC refers to the animals treated with WSG1002. Values were expressed as means±SEM. Unpaired student's t-tests were used to make statistical comparisons of IC₅₀ values of the relaxation curves (**P≦0.01);

FIG. 12 is a graph showing quantitative measurement of eNOS staining in endothelial cells of small pulmonary arteries of normoxic and chronically hypoxic rat lungs with or without treatment with WSG1002. n=4-6*P<0.05.

### DETAILED DESCRIPTION OF THE INVENTION

Compounds used in accordance with the present invention were synthesised according to known procedures as set out in Al-Hassan et. al., J. Chem. Soc. Perkin Trans I, 1985, pp 1645-1659; Cameron et. al., J. Chem. Soc. Perkin Trans. I, 1985, pp 2133-2143; and Al-Hassan et. al. J. Chem. Soc. Perkin Trans. I, 1985, pp 2145-2150 all of which are incorporated herein by reference.

Table 1 lists a number of compounds which were synthesised and tested according to the following procedures.

### Effects on Rat Pulmonary Artery and Aorta Relaxation

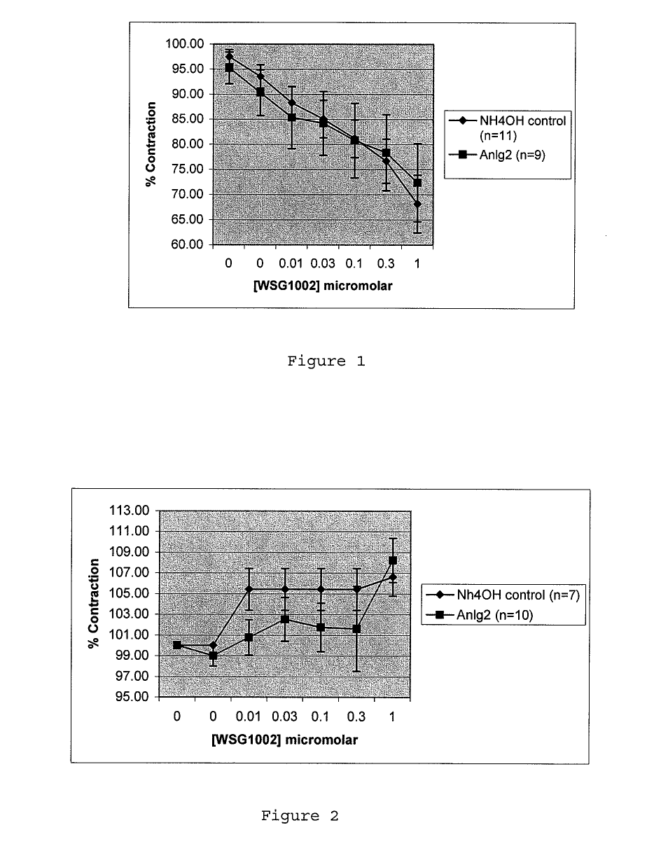

Rings (5 mm long) of rat pulmonary artery and aorta, were supported on a pair of intraluminal wires at 1 gm tension for one hour and then contracted with their EC₅₀ phenylephrine concentration, which for pulmonary artery was 3.6×10⁻⁸ M (derived from preliminary experiments) and for aorta was 11×10⁻⁸ M (from the previously published literature). The rings were then relaxed with carbachol (10 μM). If the relaxation to carbachol was >75%, then the tissues were used to generate dose responses by addition of tetrahydrobiopterin (BH₄) or the analogues. Stock BH4 was stored at −20° C. and the dilutions were prepared using saline just before adding the drug into the bath. Solutions of analogues were stored at 4° C. Cumulatively increasing concentrations of the analogues were added to the bath and the extent of relaxation was measured when it had stabilised. Another ring from the same rat was treated in parallel with the solvent for the analogue. The solvent used was NH₄OH. The results are shown graphically in FIGS. 1 and 2.

### Effects on BH4 Depleted Rat Pulmonary Artery and Aorta Relaxation

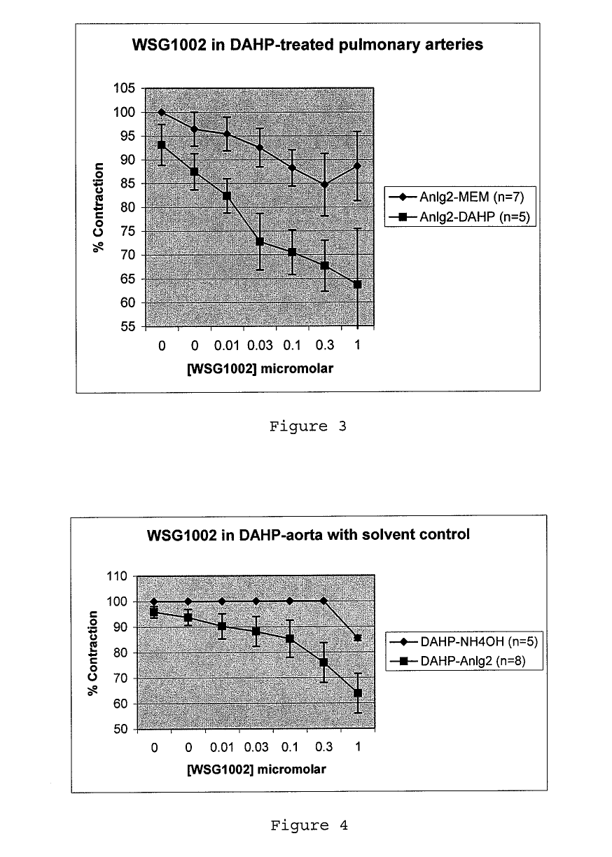

Rat pulmonary artery or aorta rings (as above) were placed in 15 ml tubes containing 2-3 ml of MEM (minimal essential medium) solution containing 0.1% bovine serum albumin, 100 U/ml penicillin and 100 μM/ml streptomycin. Some of the tubes in addition received diamino-hydroxy-pyrimidine (DAHP) 10 M, an inhibitor of GTP cyclohydrolase, the rate-limiting step in tetrahydrobiopterin synthesis. The tubes were incubated at 37° C. for six hours. This treatment has been demonstrated to deplete endogenous tetrahydrobiopterin to 4% of the control level in canine basilar artery. After the incubation with DAHP or the control incubation with the medium (MEM) alone, the rings were removed and then their relaxation in response to the analogue (or solvent NH₄OH) was tested as above. Dose response curves of DAHP-treated tissues were compared to the control tissues treated in MEM alone for 6 hrs. The results are shown graphically in FIGS. 3 and 4.

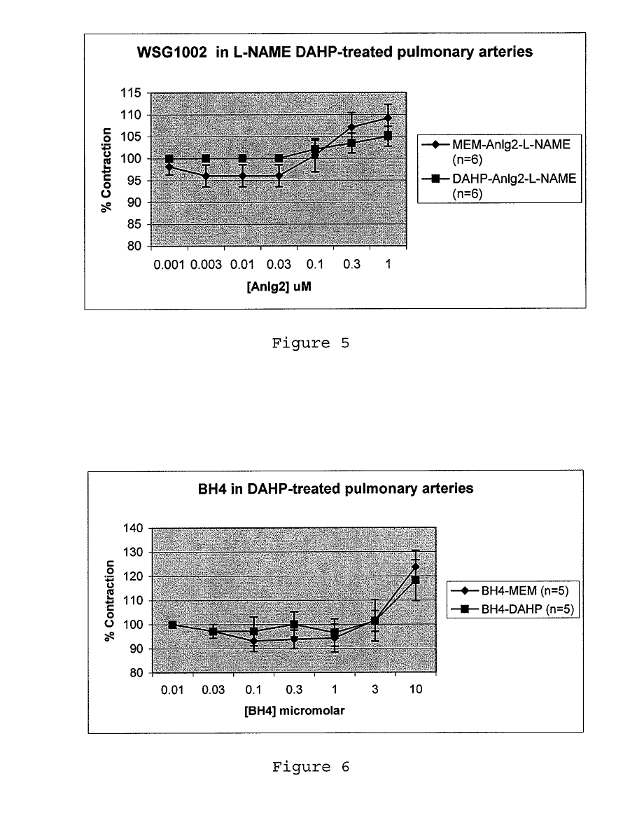

Further experiments demonstrated that the arterial relaxation was caused by NO formation, since this action was inhibited by the NO synthase blocker, L-NAME (FIG. 5). Thus WSG1002 is acting to enhance the ability of the endothelium to form NO and is not acting merely as a NO donor.

The above procedures were used to mimic cardiovascular disease, by treating aorta and pulmonary artery rings with an inhibitor of the enzyme GTP cyclohydrolase, which is the rate-limiting step in the formation of tetrahydrobiopterin by cells. Treatment with this inhibitor, DAHP, for 6 hours reduced endogenous tetrahydrobiopterin levels in arteries to 4% of the normal content. In DAHP-treated artery rings, addition of the compound having reference code WSG1002 caused a significant and physiologically important vasodilatation. This was found in both pulmonary artery and aorta (FIGS. 3 and 4). In contrast, no action was found in arteries that had not been depleted of their tetrahydrobiopterin, thus demonstrating that this action is specific for diseased arteries in this model. From this result, WSG1002 appears to be able to cross the cell membrane and enter the cells in the artery, and to substitute for the normal cofactor (tetrahydrobiopterin) in the formation of nitric oxide. WSG1002 was superior to tetrahydrobiopterin itself. Addition of tetrahydrobiopterin did not produce any relaxation of rat aorta or rat pulmonary artery rings (FIG. 6). The only effect seen was a contraction—the opposite to the action of WSG1002—at the highest concentration of tetrahydrobiopterin. The superior action of WSG1002 in comparison with tetrahydrobiopterin is probably due to improved cell permeation, stability and affinity for the target site. WSG1002 has an EC₅₀ of approximately 10 nM.

### Effects on Whole Cells

The arterial assay is sensitive but slow to perform, and therefore a wider range of compounds was investigated in whole cell preparations. Tests have been carried out using endothelial cells that produce eNOS and stimulated macrophages that produce iNOS.

### Macrophage Assay

This is a cell based assay with lipopolysaccharide stimulated macrophages in which nitric oxide is produced by the action of iNOS.

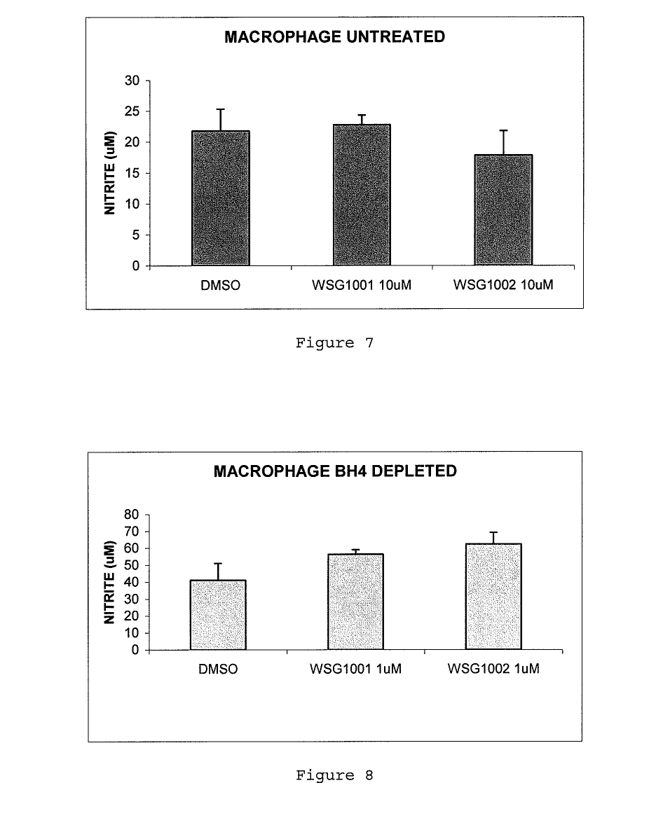

Primary cultures of macrophage cells isolated from mouse femur were produced over a 7 day period. At this stage the cells were seeded into 96-well culture plates at a density of 2×10⁵ cells per well. After further incubation for 24 hrs, the well media was changed and the relevant treatment was introduced. The cells were then incubated for a further 72 hrs before 50 μl was removed from each well and combined with 100 μl of Greiss reagent for NO₂ determination. All treatments were performed in triplicate. In each plate, 3 wells were designated control i.e. media change only-no LPS. These samples represent basal NO₂ output over 72 hrs. 3 wells were used for LPS (0.5 μg/ml) stimulation alone and 3 wells were used for LPS+0.05% DMSO. This DMSO concentration was present in all subsequent analogue treated wells. All analogues were tested at 1.0 and 10 μM. The results are shown in FIG. 7.

In another set of experiments, the assay was repeated with the inclusion of a step to deplzete endogenous tetrahydrobiopterin as in the artery ring studies. DAHP pre-treatment was employed to deplete the macrophage cells of endogenous tetrahydrobiopterin. In these experiments, DAHP (10⁻² M) was added to the cells for 24 hrs prior to seeding into 96-well plates and for 24 hrs following the seeding procedure. Thereafter, DAHP was present throughout the analogue treatment period of 72 hrs. The results are shown in FIG. 8.

### Endothelial Cell Assay

Endothelial cells were isolated from porcine pulmonary artery and grown to confluency in T75 flasks. Subsequently, the cells were seeded into 6-well plates. At confluency, the medium (1 ml/well) was changed and the relevant treatment introduced to all wells in each plate (one treatment/plate). The cells were incubated for a further 7-day period and the experiment was terminated. The media from the wells of respective plates was combined (˜6 mls) and evaporated to approximately 500 μl (˜10-fold). A 50 μl aliquot was removed and any NO₃ was converted to NO₂ using the NO₃ conversion method (Calbiochem Cat No 482702). These treated samples were then assayed using the Greiss reaction as described above giving the content of the combination of (NO₂+NO₃).

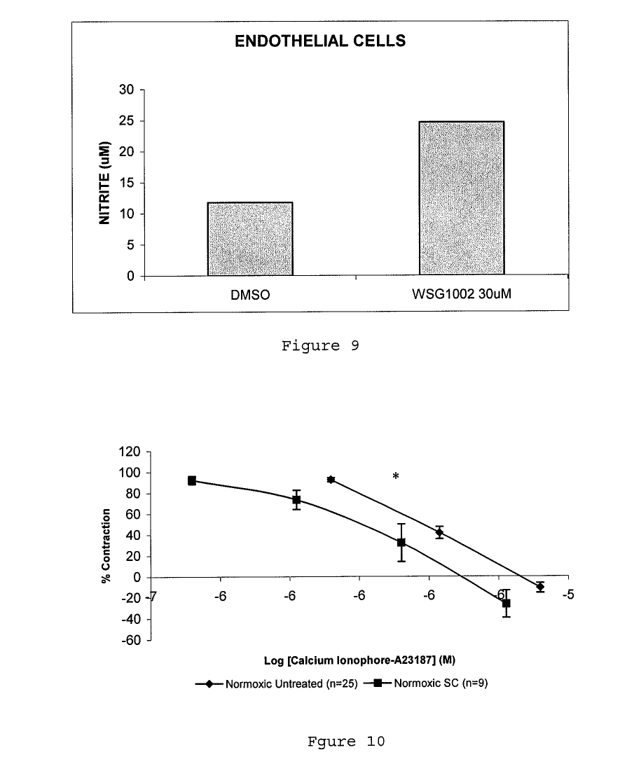

WSG1002, as a representative compound, was incubated with a preparation of endothelial cells over seven days in order to accumulate sufficient nitric oxide for assay as described above. Controls included vehicle (0.5% DMSO) and L-NAME (an established NOS inhibitor). WSG1002 caused a doubling of nitric oxide compared with vehicle control. These results, which are shown in FIG. 9, confirm the ability of WSG 1002 to act as a stimulator of nitric oxide production.

Whilst the use of a GTP cyclohydrolase I inhibitor such as DAHP can reduce tetrahydrobiopterin levels sufficiently to establish effects of test compounds, as shown in the tissue assays described above, it cannot completely remove endogenous tetrahydrobiopterin. Macrophages were initially depleted of tetrahydrobiopterin by incubation with DAHP. Under these conditions, it was possible however to measure both promotion and inhibition of nitric oxide production in these cells. The activity shown in cells after treatment with DAHP can be ascribed to residual endogenous tetrahydrobiopterin in the cells. FIG. 8 shows the behaviour of WSG1001 and WSG1002 in this assay. The increases were statistically significant.

The activity for all compounds tested is summarised in Table 1 in which P=promotes NOS activity, when tested at 1 microM.

TABLE 1

| | | | | Mol. | Log | Clog | INOS effect |
|---|---|---|---|---|---|---|---|
| Reg. No. | Structure | Name | Formula | Wt. | P | P | *H155 |
| wsg1001 | 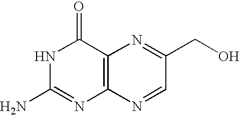 | 2-Amino-6-hydroxymethyl-3H-pteridin-4-one | C7H7N5O2 | 193.16 | −1.02 | −2.64 | P |
| wsg1002 | 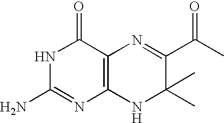 | 6-Acetyl-2-amino-7,7-dimethyl-7,8-dihydro-3H-pteridin-4-one | C10H13N5O2 | 235.24 | −1.41 | 0.03 | P |
| wsg1003 | 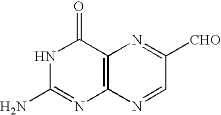 | 2-Amino-4-oxo-3,4-dihydro-pteridine-6-carbaldehyde | C7H5N5O2 | 191.15 | −0.70 | −1.67 | P |
| wsg1004 | 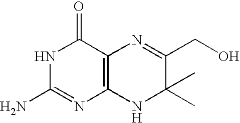 | 2-Amino-6-hydroxymethyl-7,7-dimethyl-7,8-dihydro-3H-pteridin-4-one | C9H13N5O2 | 223.23 | −1.74 | −0.82 | P |
| wsg1005 | 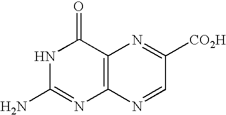 | 2-Amino-4-oxo-3,4-dihydro-pteridine-6-carboxylic acid | C7H5H5O3 | 207.15 | −0.89 | −0.97 | P |
| wsg1006 | 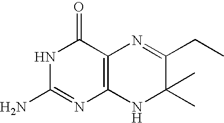 | 2-Amino-6-ethyl-7,7-dimethyl-7,8-dihydro-3H-pteridin-4-one | C10H15N5O | 221.26 | −0.39 | 0.63 | P |
| wsg1007 | 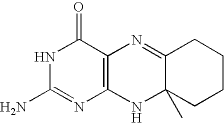 | 2-Amino-9a-methyl-6,7,8,9,9a,10-hexahydro-3H-benzo[g]pteridin-4-one | C11H15N5O | 233.37 | −0.40 | 0.49 | P |
| wsg1008 | 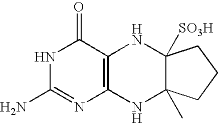 | 6-Amino-3a-methyl-8-oxo-1,2,3,3a,4,7,8,9-octahydro-4,5,7,9-tetraaza-cyclopenta[b]naphthalene-9a-sulfonic acid | C10H15N5O4S | 301.32 | −1.11 | −0.92 | P |
| wsg1009 | 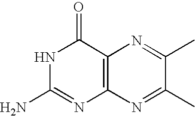 | 2-Amino-6,7-dimethyl-3H-pteridin-4-one | C8H9N5O | 191.08 | 0.54 | −0.65 | P |
| wsg1010 | 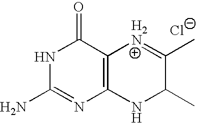 | 2-Amino-6,7-dimethyl-4-oxo-3,4,5,6,7,8-hexahydro-pteridin-5-ium Cl— | C8H14ClN5O | 231.68 | −0.90 | −0.20 | P |
| wsg1011 | 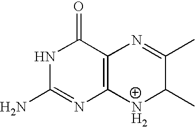 | 2-Amino-6,7-dimethyl-4-oxo-3,4,7,8-tetrahydro-pteridin-8-ium Cl— | C8H12ClN5O | 194.21 | −0.91 | −1.51 | P |
| wsg1012 | 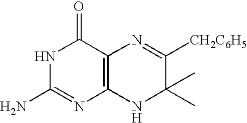 | 2-Amino-6-benzyl-7,7-dimethyl-7,8-dihydro-3H-pteridin-4-one | C15H17N5O | 283.33 | 0.80 | 1.80 | P |
| wsg1014 | 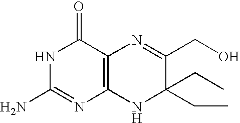 | 2-Amino-7,7-diethyl-6-hydroxymethyl-7,8-dihydro-3H-pteridin-4-one | C11H15N5O2 | 251.28 | −0.77 | 0.24 | P (weak) |
| wsg1016 | 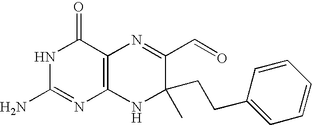 | 2-Amino-7-methyl-4-oxo-7-phenethyl-3,4,7,8-tetrahydro-pteridine-6-carbaldehyde | C16H17N5O2 | 311.34 | 0.91 | 1.10 | P (weak) |
| wsg1017 | 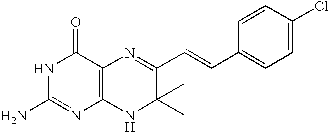 | 2-Amino-6-[2-(4-chloro-phenyl)-vinyl]-7,7-dimethyl-7,8-dihydro-3H-pteridin-4-one | C16H16ClN5O | 329.78 | 1.76 | 3.08 | P (strong) |
| wsg1018 | 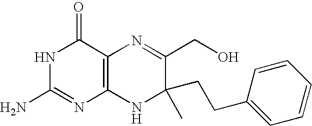 | 2-Amino-6-hydroxymethyl-7-methyl-7-phenethyl-7,8-dihydro-3H-pteridin-4-one | C16H19N5O2 | 313.35 | 0.35 | 1.33 | P |
| wsg1019 | 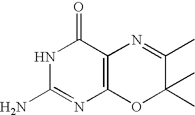 | 2-Amino-6,7,7-trimethyl-3,7-dihydro-pyrimido[4,5-b][1,4]oxazin-4-one | C9H12N4O2 | 208.22 | −0.96 | −0.20 | P (weak) |

*all compounds were tested at 1 micoM for effects on iNOS in macrophages: P = 20-50% change; “strong” = greater than 50% change; “weak” = less than 20% change

Effects on Normotensive and Pulmonary Hypertensive Rats

Rats were maintained in a chamber containing atmospheric air at 550 mbar for 14 days. These animals were confirmed to have pulmonary hypertension, as demonstrated by right ventricular hypertrophy and increased lung perfusion pressure. Age-matched control animals were maintained at normal atmospheric pressure, and these did not have pulmonary hypertension.

A group of normotensive and a group of pulmonary hypertensive rats received daily subcutaneous injections of WSG1002 in a dose of 14.1 mg/kg/day during the time that they were in the hypoxic chamber. Other groups of normotensive and pulmonary hypertensive rats had no drugs and are used as control comparison groups. These studies used lungs from the four groups of rats (1) pulmonary hypertensive and treated with WSG1002, (2) normotensive and treated with WSG1002, (3) pulmonary hypertensive and not treated with WSG1002 (4) normotensive and not treated with WSG1002.

Effects on Calcium Ionophore-Induced Pulmonary Vasodilation—Administration of WSG1002 to Pulmonary Hypertensive Rats Improves Pulmonary Endothelium function:

The rats lungs were perfused at constant flow through the pulmonary artery with measurement of perfusion pressure and maintained at 37° C. The perfusate was 30 ml of Krebs-Henseleit solution supplemented with albumin and flurbiprofen and was recirculated. The lungs were also ventilated with either normoxic gas (20% O₂, 5% CO₂ and 75% N₂) or hypoxic gas (0% O₂ and 100% N₂) during hypoxic challenge. The perfusate was equilibrated with the same gas mixture as was used for ventilation.

The lungs were precontracted with the thromboxane A₂ mimetic, 9,11-dideoxy-11_{α},9_{α}-epoxymethanoprostaglandin F_{2α}, (U46619, 3×10⁻⁵ M) given as a bolus dose via an injection port. Then the gas supply was changed from 20% O₂ to 0% O₂ for 10 minutes to record hypoxic vasoconstriction. Following return to 20% O₂, the lungs were precontracted again with a bolus dose of U46619 and relaxation responses to the endothelium-dependent vasorelaxant, calcium ionophore were measured by adding it in increasing doses to the perfusate. The data was plotted as concentration-response curves from which IC₅₀ values were calculated using Biograph program by fitting the curves to the Hill equation y=RMax/(1+(x/EC₅₀)̂P).

### Results

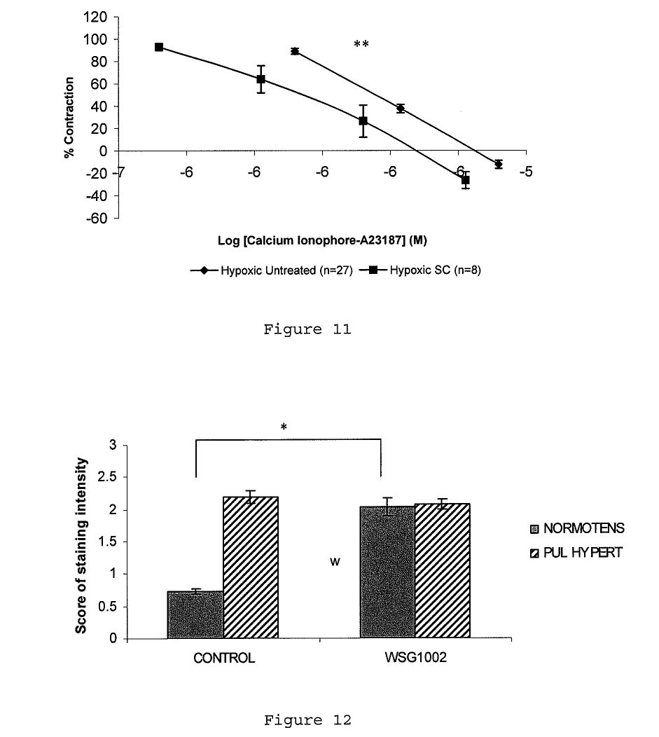

Pulmonary vasodilation produced by the endothelium-dependent vasorelaxant calcium ionophore, were greater in rats that had received treatment with WSG1002, compared to untreated rats (FIGS. 10 and 11). WSG1002 improved endothelium-dependant relaxation in both pulmonary hypertensive and normotensive rats. The IC₅₀ values for calcium ionophore in pulmonary hypertensive rats was reduced from 1.7 μM to 1.2 μM (P≦0.01) and in the normotensive control group the IC₅₀ was reduced from 1.5 μM to 1.1 μM (P≦0.05).

Effect of Administration of WSG1002 to Pulmonary Hypertensive Rats—eNOS in Pulmonary Vascular Endothelium is Increased:

The rat lungs were fixed with 4% formaldehyde, wax embedded, and 3 μm thick sections were cut. The sections were mounted on silanated slides, rehydrated and treated with 0.3% H₂O₂ to block endogenous peroxidase activity. Antigens were unmasked by treatment in a microwave oven. The sections were then treated with 20% normal goat serum followed by incubation with anti-eNOS mouse monoclonal antibody at a dilution of 1:2000 for 1 hr at room temperature. Then biotinylated secondary antibody was added to the sections followed by streptavidin-horse raddish peroxidase complex and then 3,3-diaminobenzidinetetrahydrochloride. The sections were then counter-stained with haematoxylin to locate the cell nuclei. The slides were then dehydrated to xylene and mounted in DPX.

Slides were coded and viewed under magnification 400× to identify pulmonary arteries (≦200 μm diameter). In the endothelium, the intensity of staining was quantified on a scale of 0-3, where 0=no staining or the same as the negative control, 1=faint staining or staining in a few target cells, 2=moderate staining in most target cells, 3=the maximum staining observed with that antibody in the positive control slides.

### Results

The endothelium of small pulmonary arteries stained weakly for eNOS in normotensive (control) rats, however this was markedly increased in pulmonary hypertensive rats (FIG. 12). Rats that had been treated with WSG1002 had increased staining for eNOS in normotensive rats, and there was no further increase when the rats were made pulmonary hypertensive (FIG. 12).

### REFERENCES

1. Presta A, Siddhanta U, Wu, C, Sennequier N, Huang L, Abu-Soud HM, Erzurum S, Stuehr D J (1998) Comparative functioning of dihydro- and tetrahydropterins in supporting electron transfer, catalysis and subunit dimerization in inducible nitric oxide synthase. Biochemistry 37, 298-310.
2. Guzik T J; Mussa S; Gastaldi D; Sadowski J; Ratnatunga C; Pillai R; Channon K M (2002) Mechanisms of increased vascular superoxide production in human diabetes mellitus Role of NAD(P)H oxidase and endothelial nitric oxide synthase. Circulation 105, 1656-1662.
3. Tiefenbacher C P, Bleeke T, Vahl C, Amann k, Vogt A, Kubler W (2000) Endothelial dysfunction of coronary resistance arteries is improved by tetrahydrobiopterin in atherosclerosis. Circulation 102, 2172-2179.
4. Koller A; Bagi ZS (2003) Reduced level of tetrahydrobiopterin is responsible for the lack of nitric oxide mediation of flow-dependent arteriolar dilation in diabetes mellitus. Eur Heart J 24, 214-214.
5. Pannirselvam M; Verma S; Anderson T J; Triggle C R (2002) Cellular basis of endothelial dysfunction in small mesenteric arteries from spontaneously diabetic (db/db−/−) mice: role of decreased tetrahydrobiopterin bioavailability. Br J Pharmacol 136, 255-263.
6. Alp N J; Mussa S; Khoo J; Cai S J; Guzik T; Jefferson A; Goh N; Rockett K A; Channon K M (2003) Tetrahydrobiopterin-dependent preservation of nitric oxide-mediated endothelial function in diabetes by targeted transgenic GTP-cyclohydrolase I overexpression. J Clin Invest 112, 725-735.
7. Vásquez-Vivar J, Duquaine D, Whitsett J, Kalyanaraman B, Rajagopalan S (2002) Altered tetrahydrobiopterin metabolism in atherosclerosis. Implications for use of oxidised tetrahydrobiopterin analogues and thiol antioxidants. Atrerioscler Thromb Vasc Biol 22, 1655-1661.
8. Ozaki M; Kawashima S; Yamashita T; Namiki M; Hirase T; Inoue N; Hirata K; Yokoyama M (2002) Supplementation of tetrahydrobiopterin inhibits atherosclerosis in apolipoprotein E-deficient mice. Circulation 106, 348.
9. Bagi Z; Koller A (2003) Lack of nitric oxide mediation of flow-dependent arteriolar dilation in type I diabetes is restored by sepiapterin. J Vasc Res 40, 47-57.
10. Katusic Z S (2001) Vascular endothelial dysfunction: does tetrahydrobiopterin play a role? Am J Physiol 281, H981-H986.
11. Pannirselvam M; Anderson T J; Triggle C R(2003) Endothelial cell dysfunction in type I and II diabetes: The cellular basis for dysfunction. Drug Dev Res 58, 28-41.
12. Shinozaki K; Kashiwagi A; Masada M; Okamura T (2003) Molecular mechanisms of endothelial dysfunction in the insulin-resistant state: the roles of abnormal pteridine metabolism. Pteridines 14, 13-16.
13. Heitzer T, Krohn K, Albers S, Meinertz T (2000) Tetrahydrobiopterin improved endothelium-dependent vasodilation by increasing nitric oxide activity in patients with type II diabetes mellitus. Diabetologia 43, 1435-1438.
14. van Etten R W; de Koning E J P; Verhaar M C; Gaillard C A J M; Rabelink T J (2002) Impaired NO-dependent vasodilation in patients with Type II (non-insulin-dependent) diabetes mellitus is restored by acute administration of folate. Diabetologia 45, 1004-1010.
15. Ihlemann N; Rask-Madsen C; Perner A; Dominguez H; Hermann T; Kober L; Torp-Pedersen C (2003) Tetrahydrobiopterin restores endothelial dysfunction induced by an oral glucose challenge in healthy subjects. Am J Physiol 285, H875-H882.
16. Stroes E, Kastelein J, Cosentino F, Erkelens W, Wever R, Koomans H, Luscher T (1997) Tetrahydrobiopterin restores endothelial function in hypercholesterolaemia. J Clin Invest 99, 41-46.

17. Fukuda Y; Teragawa H; Matsuda K; Yamagata T; Matsuura H; Chayama K (2002) Tetrahydrobiopterin restores endothelial function of coronary arteries in patients with hypercholesterolaemia. Heart 87, 264-269.

1. A method of enhancing nitric oxide formation by nitric oxide synthase, comprising administering to a subject in need thereof a compound of formula (I): 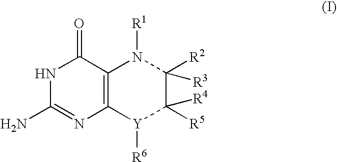
or pharmaceutically acceptable derivatives and/or salts thereof, wherein,
Y is an oxygen or a nitrogen atom;
R² and R⁵ are independently hydrogen, unsubstituted or substituted alkyl, unsubstituted or substituted alkenyl, unsubstituted or substituted aryl, hydroxyl, amino, halo, alkanoyl, alkyl carboxy, sulfonyl and hydroxyimino;
R³ and R⁴ are independently hydrogen, unsubstituted or substituted alkyl, unsubstituted or substituted alkenyl, unsubstituted or substituted aryl, hydroxyl, amino, halo, alkanoyl, alkyl carboxy, sulfonyl and hydroxyimino,
or R³ and R⁴ taken together with the ring carbons to which they are bonded, form an unsubstituted or substituted carbocyclic ring;
the dashed lines are independently a carbon-carbon single bond, wherein R¹ and R⁶ are hydrogen, or the dashed lines are independently a carbon-carbon double bond and the groups R¹ and R², and/or R⁵ and R⁶ associated with the carbon-carbon double bond are absent, with the proviso that when Y is an oxygen atom, R⁶ is absent and the dashed line attached to Y is a single bond.

2. A method according to claim 1, wherein the method is for one or more of the treatment of a disease associated with a deficiency of nitric oxide, a local deficiency of nitric oxide, a deficiency at one or more veins or arteries, and a deficiency associated with a site of damage or injury of a vein or artery.

3. A method according to claim 2, wherein said disease is selected from the group consisting of diabetes, atherosclerosis, hyperlipidaemia, arterial high blood pressure, haemostasis disorders, coronary heart disease and erectile dysfunction.

4. A method according to claim 3, wherein said disease is associated with endothelial dysfunction.

5. A method according to claim 1, wherein in formula (I), R², R³, R⁴ and R⁵ are independently an unsubstituted or substituted alkyl, alkenyl, alkanoyl or carboxy.

6. A method according to claim 1, wherein in formula (I), R², R³, R⁴ and R⁵ are independently an unsubstituted or substituted alkyl or alkenyl group having from 1 to 24 carbon atoms.

7. A method according to claim 1, wherein in formula (I), R², R³, R⁴ and
R⁵ are independently an unsubstituted or substituted C₁-C₁₀ alkyl or alkenyl group.

8. A method according to claim 6, wherein said alkyl or alkenyl group is substituted with a substituent chosen from hydroxy, amino, carboxy, halo, sulfonyl and unsubstituted or substituted aryl.

9. A method according to claim 1, wherein in formula (I), R², R³, R⁴ and R⁵ are independently an alkanoyl group.

10. A method according to claim 9, wherein the alkanoyl group is a ketone or aldehyde.

11. A method according to claim 1, wherein R³ and R⁴ form a 5 to 7 membered saturated or unsaturated carbocyclic ring with the carbons to which they are attached.

12. A method according to claim 1, wherein, R⁴ and R⁵ are independently either hydrogen or lower alkyl.

13. A method according to claim 1, wherein, both of said dashed lines are double bonds.

14. A method according to claim 1, wherein, both of said dashed lines are double bonds and R¹, R², R⁵, and R⁶ are absent, R² is hydroxymethyl and R⁴ is hydrogen.

15. A method according to claim 1, wherein, one of said dashed lines is a double bond and R¹ and R² are absent, R³ is acetyl, R⁴ and R⁵ are methyl, and R⁶ is hydrogen.

16. A method according to claim 1, wherein one of the dashed lines is a double bond and R¹ and R² are absent, R³ is 2-(4-chlorophenyl)-vinyl, R⁴ and R⁵ are methyl, and R⁶ is hydrogen.

17. A method according to claim 1 wherein, in said subject in need thereof, there is a local deficiency of nitric oxide.

18. A method according to claim 17, wherein the deficiency is at one or more veins or arteries.

19. A method according to claim 17, wherein the deficiency is at a site of damage or injury of a vein or artery.

20. A method for the treatment of a disease associated with a deficiency of nitric oxide, comprising administering to a subject in need thereof, a compound according to formula (I): 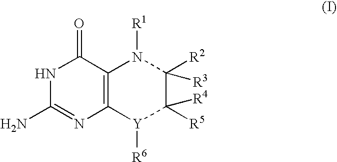
or pharmaceutically acceptable derivatives and/or salts thereof, wherein,
Y is an oxygen or a nitrogen atom;
R² and R⁵ are independently hydrogen, unsubstituted or substituted alkyl, unsubstituted or substituted alkenyl, unsubstituted or substituted aryl, hydroxyl, amino, halo, alkanoyl, alkyl carboxy, sulfonyl and hydroxyimino;
R³ and R⁴ are independently hydrogen, unsubstituted or substituted alkyl, unsubstituted or substituted alkenyl, unsubstituted or substituted aryl, hydroxyl, amino, halo, alkanoyl, alkyl carboxy, sulfonyl and hydroxyimino,
or R³ and R⁴ taken together with the ring carbons to which they are bonded, form an unsubstituted or substituted carbocyclic ring;
the dashed lines are independently a carbon-carbon single bond, wherein R¹ and R⁶ are hydrogen, or the dashed lines are independently a carbon-carbon double bond and the groups R¹ and R², and/or R⁵ and R⁶ associated with the carbon-carbon double bond are absent, with the proviso that when Y is an oxygen atom, R⁶ is absent and the dashed line attached to Y is a single bond.

21. A method according to claim 20, wherein, said disease is selected from the group consisting of diabetes, atherosclerosis, hyperlipidaemia, arterial high blood pressure, haemostasis disorders, coronary heart disease and erectile dysfunction.

22. A method according to claim 20, wherein said disease is associated with endothelial dysfunction.

23. A method according to claim 20, wherein a local amount of nitric oxide is selectively increased thereby.

24. A method according to claim 20, wherein nitric oxide synthase is activated thereby.

25. A method according to claim 20, wherein, the subject to be treated is a human or non-human animal.
